(19) United States
(12) Patent Application Publication
(10) Pub. No.: US 2023/0372365 A1
(43) Pub. Date: Nov. 23, 2023

(54) METHODS OF TREATING FIBROMYALGIA WITH NEUROACTIVE STEROIDS

(71) Applicants: Andrew D. LEVIN, Newton, MA (US); Danielle FELDMAN, Boston, MA (US); Eliem Therapeutics (UK) Ltd, Altrincham (GB)

(72) Inventors: Andrew D. Levin, Newton, MA (US); Danielle Feldman, Boston, MA (US)

(21) Appl. No.: 18/027,758

(22) PCT Filed: Sep. 30, 2021
(86) PCT No.: PCT/US2021/052857

§ 371 Date: Mar. 22, 2023

Related U.S. Application Data
(60) Provisional application No. 63/086,467, filed on Oct. 1, 2020.

(51) Int. Cl. A61K 31/58; A61K 45/06

(52) U.S. Cl. CPC A61K 31/58; A61K 45/06

(57) ABSTRACT

The invention relates to methods of treating fibromyalgia in a human subject by providing the steroid of Formula (I):

### FIELD OF THE INVENTION

The invention relates to methods of treating fibromyalgia by providing compositions containing the compound of Formula (I).

### BACKGROUND

Fibromyalgia is among the most frequent causes or chronic widespread pain globally, thought to affect 2% to 5% of the adult population. The pain caused fibromyalgia can be severe and known to migrate to all parts of the body with varying severity. In addition to chronic pain, fibromyalgia is characterized fatigue, depression, anxiety, stiffness, sleep disorders, difficulty concentrating, memory loss, headaches, vision problems numbness, temporomandibular joint syndrome, ringing in the ears, digestive problems, including irritable bowel and bladder disorders, restless leg syndrome, skin sensitives and rashes, and dry eyes and mouth, among other symptoms. The fatigue associated with fibromyalgia can itself be debilitations, interfering with the ability for individuals to complete day-to-day tasks. This is often compounded by the sleep abnormalities, vision problems, and difficulties with concentration and memory associated with the condition. As a result, fibromyalgia debilitates tens of millions of people around the world.

Unfortunately, the causes of fibromyalgia are still poorly understood. Although it is thought that chronic pain from fibromyalgia may results from alternations in neurotransmitters and neuro-chemical processing of sensory signals in the central nervous system, thus lowering the threshold for pain and amplification of normal sensory signals, the full etiology is still unknown. As a result, although an array of medications have been proposed to treat fibromyalgia, no pharmacological agent or combination of agents has been ultimately effective in the treatment of fibromyalgia. Approved drugs for the treatment of fibromyalgia, including serotonin reuptake inhibitor antidepressants, such as duloxetine (sold under the brand name CYMBALTA by Eli Lilly and Company), serotonin-norepinephrine reuptake inhibitors, such as milnacipran (sold under the brand name SAVELLA by Allergan), and anticonvulsants, such as pregabalin (marketed under the name LYRICA by Pfizer Inc.) and gabapentin (sold under the brand name NEURONTIN by Pfizer Inc.), each cause their own severe side effects, many of which are symptoms of fibromyalgia itself, including difficulty sleeping, headaches, dizziness, blurred vision, abnormal eye movements, trouble concentrating, constipation, diarrhea, nausea, vomiting, dry mouth, sweating, tiredness, loss of appetite, flushing, swelling in hands or feet, weight changes, decreased sex drive, and impotence among others. Moreover, pharmacological agents proposed for the treatment of fibromyalgia have shown limited long-term efficacy for fibromyalgia beyond some symptomatic alleviation.

### SUMMARY

The invention provides methods for treating fibromyalgia in a human subject by providing a compound having the structure of Formula (I):

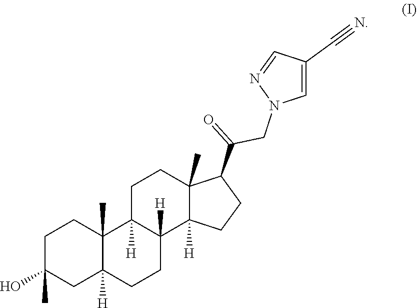

The compound of Formula (I) is an allosteric modulator of receptors for the neurotransmitter γ-aminobutyric acid (GABA), for example the GABA_{A} receptor. The invention recognizes that fibromyalgia, understood to be influenced by changes in the central nervous system, can be ameliorated by providing the compound of Formula (I). Therefore, the invention provides methods of treating fibromyalgia related disorders using compositions that contain therapeutically effective amounts of the compound of Formula (I).

In an aspect, the invention provides methods of treating fibromyalgia by providing to a subject suffering from fibromyalgia a composition containing a compound of Formula (I):

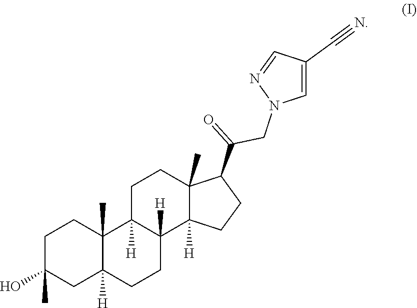

Fibromyalgia may be associated with one or more symptoms. For example, the condition may be chronic pain, widespread pain, allodynia, hyperalgesia, fatigue, depression, anxiety, stiffness, sleep disorders, difficulty concentrating, memory loss, headaches, vision problems numbness, temporomandibular joint syndrome, ringing in the ears, digestive problems, including irritable bowel and bladder disorders, restless leg syndrome, skin sensitives and rashes, and dry eyes and mouth.

Treating fibromyalgia may comprise treating chronic pain. Treating fibromyalgia may comprise treating fatigue. Treating fibromyalgia may comprising treating one or more additional symptoms of fibromyalgia selected from the group consisting of chronic pain, widespread pain, allodynia, hyperalgesia, fatigue, depression, anxiety, stiffness, sleep disorders, difficulty concentrating, memory loss, headaches, vision problems numbness, temporomandibular joint syndrome, ringing in the ears, digestive problems, including irritable bowel and bladder disorders, restless leg syndrome, skin sensitives and rashes, and dry eyes and mouth.

In aspects of the invention, treating fibromyalgia comprises reducing fibromyalgia-associated pain by at least 30%, at least 40%, at least 50%, at least 60%, at least 70%, at least 80%, or at least 90%. In aspects of the invention, treating fibromyalgia comprises eliminating pain associated with fibromyalgia or preventing pain associated with fibromyalgia.

The compound of Formula (I) may also be provided in combination with one or more compound useful for treating symptoms of fibromyalgia. In aspects of the invention, the method comprises administering one or more additional active agents selected from the group consisting of antidepressants, anti-inflammatories, muscle relaxants, antibiotics, mood stabilizers, antipsychotics, serotonin receptor antagonists, serotonin receptor agonists, pain relievers, stimulants, NMDA receptor ligands, serotonin reuptake inhibitor antidepressants, serotonin-norepinephrine reuptake inhibitors, and anticonvulsants.

Fibromyalgia is understood to present more commonly in women. Accordingly, the subject may be a human female. The human female may or may not be pregnant, may or may not have been previously pregnant, may or may not be fertile, may or not be pre-menopausal, or may or may not be post-menopausal.

The composition may be provided by any suitable route or mode of administration. For example, the composition may be provided orally, intravenously, enterally, parenterally, dermally, buccally, topically (including transdermally), by injection, nasally, pulmonarily, and with or on an implantable medical device (e.g., stent or drug eluting stent or balloon equivalents).

The composition may be provided to the subject according to a dosing schedule. The composition may be provided in one dose per day. The composition may be provided multiple doses per day. The composition may be provided in two, three, four, five, six, or more doses per day.

The composition may be provided by any suitable route or mode of administration. For example, the composition may be provided orally, intravenously, enterally, parenterally, dermally, buccally, topically (including transdermally), by injection, nasally, pulmonarily, and with or on an implantable medical device (e.g., stent or drug eluting stent or balloon equivalents).

The composition may be provided to the subject according to a dosing schedule. The composition may be provided in one dose per day. The composition may be provided multiple doses per day. The composition may be provided in two, three, four, five, six, or more doses per day. In another aspect, the invention provides uses of a compound of Formula (I) for making a medicament for treating a female health condition related to a sex hormone.

In another aspect, the invention provides uses of a compound of Formula (I) for making a medicament for treating a subject suffering from fibromyalgia. The medicament may be for treatment of symptoms described above. The medicament may be provided by a route or mode of administration described above. The medicament may be provided according to a dosing schedule described above.

In another aspect, the invention provides use of a compound of Formula (I) for the treatment of a subject suffering from fibromyalgia. The use may be for treatment of one of the symptoms described above. The compound may be used by a route or mode of administration described above. The compound may be used according to a dosing schedule described above.

### DETAILED DESCRIPTION

The present invention provides methods of treating a fibromyalgia, the method comprising providing to a subject having fibromyalgia a compound of Formula (I):

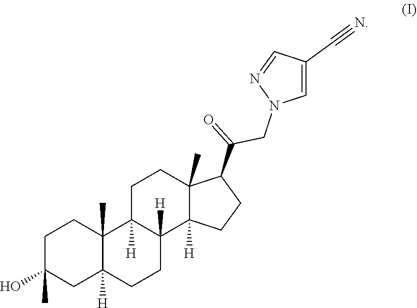

The compound of Formula (I), referred to as ATN-10155, has the IUPAC name 1-[2-[(3R,5S,8R,9S,10S,13S,14S,17S)-3-hydroxy-3,10,13-trimethyl-1,2,4,5,6,7,8,9,11,12,14,15,16,17-tetradecahydrocyclopenta[a]phenanthren-17-yl]-2-oxoethyl]pyrazole-4-carbonitrile and is disclosed in International Patent Publication No. WO 2016/061527, the contents of which are incorporated herein by reference.

The compound of Formula (I) is an allosteric modulator of receptors for the neurotransmitter γ-aminobutyric acid (GABA), for example the GABA_{A} receptor. The invention recognizes that fibromyalgia, understood to be influenced by changes in the central nervous system, can be ameliorated by providing the compound of Formula (I). Therefore, the invention provides methods of treating fibromyalgia using compositions that contain therapeutically effective amounts of the compound of Formula (I).

Without wishing to be bound by theory or mechanisms of action, it is believed that the compound of Formula (I) is a GABA_{A} receptor positive allosteric modulator. It is believed that providing the compound of Formula (I) may overcome fluctuations and stabilize GABA_{A} receptor signaling. For example, the compound of Formula (I) may be provided in amounts to GABA_{A} receptors to control signaling of such receptors. In that manner, the compound of Formula (I) may provide stimulation to the GABA_{A} receptor. Stabilization of GABA_{A} receptor signaling may be achieved by providing a continuous dose of the compound of Formula (I). Alternatively, GABA_{A} receptor signaling may be stabilized by providing intermittent doses of the compound of Formula (I).

The compound of Formula (I) is thought to selectively bind the GABA_{A} receptor at a site that is distinct from those of barbiturates and benzodiazepines. Moreover, unlike benzodiazepines, which bind exclusively at synaptic GABA_{A} receptors to mediate short bursts of ‘phasic’ inhibitor, the compound of Formula (I) is thought to bind at extra synaptic GABA_{A} receptors to mediate ‘tonic’ inhibition, which regulates network-level excitability and acts on a longer timescale. Modulating tonic inhibition is relevant to multiple disease states, including seizure and mood disorders.

The side-effects of effective doses of the compound of Formula (I) are believed to be limited. The abuse potential of effective doses of the compound of Formula (I) are believed to be limited.

### Definitions

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

As used herein, a “pure isomeric” compound or “isomerically pure” compound is substantially free of other isomers of the compound. The term “pure isomeric” compound or “isomerically pure” denotes that the compound comprises at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of the compound with the specified structure. In certain embodiments, the weights are based upon total weight of all isomers of the compound.

As used herein, a “pure stereoisomeric” compound or “stereoisomerically pure” compound is substantially free of other stereoisomers of the compound. Thus, the composition is substantially free of isomers that differ at any chiral center. If the compound has multiple chiral centers, a substantial majority of the composition contains compounds having identical stereochemistry at all of the chiral centers. The term “pure stereoisomeric” compound or “stereoisomerically pure” denotes that the compound comprises at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of the compound with the specified stereochemistry. In certain embodiments, the weights are based upon total weight of all stereoisomers of the compound.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of the enantiomer. In certain embodiments, the weights are based upon total weight of all enantiomers or stereoisomers of the compound.

Compounds described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; N may be any isotopic form, including ¹⁴N and ¹⁵N; O may be in any isotopic form, including ¹⁶O and ¹⁸O; and the like.

As used herein, the terms “modulation” and “potentiation” of GABA_{A} receptor function refer to the inhibition or stimulation of GABA_{A} receptor function. A “modulator” or “potentiator” may be, for example, an agonist, partial agonist, antagonist, or partial antagonist of the GABA receptor. The “modulator” or “potentiator” may act at the active site or at an allosteric site on a GABA receptor. It may promote or inhibit ligand binding. It may facilitate or attenuate ligand-mediated, e.g., GABA-mediated, signaling.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, el al., J. Pharm. Sci. (1977) 66(1): 1-79.

As used herein, the term “isotopic variant” refers to a compound that contains unnatural proportions of isotopes at one or more of the atoms that constitute such compound. For example, an “isotopic variant” of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e., ³H, and carbon-14, i.e., ¹⁴C, are particularly useful for this purpose in view of their ease of incorporation and ready means of detection. Further, compounds may be prepared that are substituted with positron emitting isotopes, such as ¹¹C, ¹⁸F, ¹⁵O, and ¹³N, and would be useful in Positron Emission Topography (PET) studies for examining substrate receptor occupancy. All isotopic variants of the compounds provided herein, radioactive or not, are intended to be encompassed within the scope of the invention.

“Stereoisomers”: It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers”, and those that are non-superimposable mirror images of each other are termed “enantiomers.” When a compound has an asymmetric center, for example, and an atom, such as a carbon atom, is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of n electrons and an atom (usually H). For example, enols and ketones are tautomers because they are rapidly interconverted by treatment with either acid or base. Another example of tautomerism includes the aci- and nitro-forms of phenylnitromethane that are likewise formed by treatment with acid or base. Tautomeric forms may be relevant to the attainment of the optimal chemical reactivity and biological activity of a compound of interest.

A “subject” to which administration is contemplated includes, but is not limited to, a human (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

In general, the “effective amount” of a compound refers to an amount sufficient to elicit the desired biological response, e.g., to treat a CNS-related disorder, is sufficient to induce anesthesia or sedation. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, “providing” to a subject a compound or a composition containing a compound includes both providing (1) the compound itself, or a composition containing the compound itself, and (2) providing a prodrug, analog, or derivative of the compound, or a composition containing the prodrug, analog, or derivative of the compound, in which the prodrug, analog, or derivative is converted in to the compound in vivo.

As used herein, “maximum tolerated dose” means the highest dose of a compound or composition that does not cause unacceptable side effects.

### Compositions Containing the Compound of Formula (I)

The methods of the invention include providing to a subject a composition, e.g., a pharmaceutic composition, that contains a therapeutically effective amount of a compound of Formula (I):

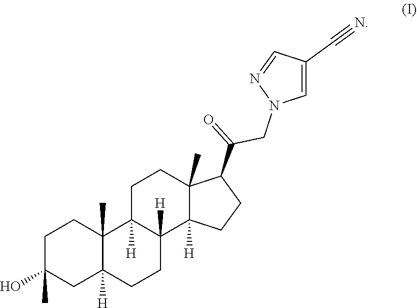

The composition may contain an isomerically pure form of a compound of Formula (I). The composition may be chemically pure, i.e., free from other molecules or chemical species. For example, the other molecule or chemical species may have a distinct chemical formula, structural formula, empirical formula, molecular formula, or condensed formula. The composition may have a defined level of chemical purity. For example, the compound of Formula (I) may be present at at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of the total amount of a mixture that includes the compound of Formula (I) and one or more distinct molecules or chemical species.

The composition may contain the compound of Formula (I) at a defined level of isomeric purity, i.e., the composition may contain the compound of Formula (I) at a level in relation to an isomeric form of the compound. For example, the compound of Formula (I) may be present at at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of the total amount of isomeric molecules that include the compound of Formula (I) and an isomer thereof.

The composition may be isomerically pure with respect to all isomers. The composition may be isomerically pure with respect to one or more particular types of isomers. The composition may be substantially free of structural isomers or a particular type of structural isomers, such as a regioisomers. The composition may be substantially free of stereoisomers or a particular type of stereoisomers, such as enantiomers or diastereomers.

The composition may contain the compound of Formula (I) at a level of isomeric purity to achieve preferential modulation of one of more subtypes of GABA_{A} receptors as compared to one or more different subtypes of GABA_{A} receptors. For example, the composition may contain the compound of Formula (I) at a level of isomeric purity to achieve preferential modulation of an α4β3δ GABA_{A} receptor as compared to an α1β2γ2 GABA_{A} receptor. The compound of Formula (I) may be present at at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of the total amount of isomeric molecules that include the compound of Formula (I) and an isomer thereof.

The composition may contain the compound of Formula (I) and be substantially free of stereoisomers. The stereoisomer may differ from Formula (I) at one, two, three, four, five, six, seven, or eight chiral centers. The stereoisomer may be a diastereomer or an enantiomer. For example, the stereoisomer may be a compound of Formulas (II) or (III):

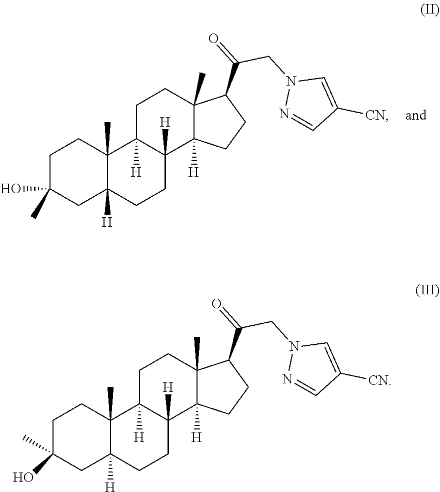

The composition may contain one or more stereoisomers of the compound of Formula (I), such as a compound of Formula (II) or (III), at less than 5%, less than 4%, less than 3%, less than 2%, less than 1%, less than 0.5%, or less than 0.1% of the total of the compound of Formula (I) and the one or more stereoisomers thereof. The composition may contain the compound of Formula (I) and one or more stereoisomer thereof at a ratio of at least 19:1, 20:1, 25:1, 30:1, 40:1, 50:1, 100:1, 200:1, 500:1, or 1000:1.

The pharmaceutical composition containing the compound of Formula (I) may be in a form suitable for oral use, for example, as tablets, troches, lozenges, fast-melts, aqueous or oily suspensions, dispersible powders or granules, emulsions, hard or soft capsules, syrups or elixirs. Compositions intended for oral use may be prepared according to any method known in the art for the manufacture of pharmaceutical compositions and such compositions may contain one or more agents selected from sweetening agents, flavoring agents, coloring agents and preserving agents, in order to provide pharmaceutically elegant and palatable preparations. Tablets contain the compounds in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. The tablets may be uncoated or they may be coated by known techniques to delay disintegration in the stomach and absorption lower down in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108; 4,166,452; and 4,265,874, the contents of which are incorporated herein by reference, to form osmotic therapeutic tablets for control release. Preparation and administration of compounds is discussed in U.S. Pat. No. 6,214,841 and U.S. Pub. No. 2003/0232877, the contents of which are incorporated herein by reference.

Formulations for oral use may also be presented as hard gelatin capsules in which the compounds are mixed with an inert solid diluent, for example calcium carbonate, calcium phosphate or kaolin, or as soft gelatin capsules in which the compounds are mixed with water or an oil medium, for example peanut oil, liquid paraffin or olive oil.

An alternative oral formulation, where control of gastrointestinal tract hydrolysis of the compound is sought, can be achieved using a controlled-release formulation, where a compound of the invention is encapsulated in an enteric coating.

Aqueous suspensions may contain the compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents such as a naturally occurring phosphatide, for example lecithin, or condensation products of an alkylene oxide with fatty acids, for example, polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the compounds in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the compounds in admixture with a dispersing or wetting agent, suspending agent and one or more preservatives. Suitable dispersing or wetting agents and suspending agents are exemplified, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavoring agents.

Syrups and elixirs may be formulated with sweetening agents, such as glycerol, propylene glycol, sorbitol or sucrose. Such formulations may also contain a demulcent, a preservative, and agents for flavoring and/or coloring. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

In certain embodiments, the formulation is not a sustained release formulation. In certain embodiments, the formulation is not injectable. In certain embodiments, the formulation does not contain particles having a D50 (volume weighted median diameter) of less than 10 microns. In certain embodiments, the formulation does not contain a polymer surface stabilizer. In certain embodiments, the formulation is not an aqueous suspension.

The composition may be formulated for administration by a particular mechanism. The composition may be formulated for oral, intravenous, enteral, parenteral, dermal, buccal, topical, nasal, or pulmonary administration. The composition may be formulated for administration by injection or on an implantable medical device (e.g., stent or drug-eluting stent or balloon equivalents).

The composition may be formulated a single daily dosage. The composition may be formulated for multiple daily dosages, e.g., two, three, four, five, six or more daily dosages.

The composition may be provided to the subject according to any dosing schedule. The composition may be provided once per day. The composition may be provided multiple times per day. The composition may be provided two times, three times, four times, five times, six times, or more per day.

### Providing a Composition Containing the Compound of Formula (I) to a Subject

Methods of the invention may include providing a composition containing a therapeutically effective amount of the compound of Formula (I) to a subject. The composition may include the compound of Formula (I) itself. Alternatively or additionally, the composition may include a prodrug, analog, or derivative of the compound of Formula (I) that is converted to the compound of Formula (I) in the body of the subject.

The composition may be provided to a subject by any suitable route or mode of administration. For example and without limitation, the composition may be provided buccally, dermally, enterally, intraarterially, intramuscularly, intraocularly, intravenously, nasally, orally, parenterally, pulmonarily, rectally, subcutaneously, topically, transdermally, by injection, or with or on an implantable medical device.

Doses may be provided at any suitable interval. For example and without limitation, doses may be provided once per day, twice per day, three times per day, four times per day, five times per day, six times per day, eight times per day, once every 48 hours, once every 36 hours, once every 24 hours, once every 12 hours, once every 8 hours, once every 6 hours, once every 4 hours, once every 3 hours, once every two days, once every three days, once every four days, once every five days, once every week, twice per week, three times per week, four times per week, or five times per week.

The dose may be provided in a single dosage, i.e., the dose may be provided as a single tablet, capsule, pill, etc. Alternatively, the dose may be provided in a divided dosage, i.e., the dose may be provided as multiple tablets, capsules, pills, etc.

The dosing may continue for a defined period. For example and without limitation, doses may be provided for at least 1 week, at least 2 weeks, at least 3 weeks, at least 4 weeks, at least 6 weeks, at least 8 weeks, at least 10 weeks, at least 12 weeks, at least 4 months, at least 5 months, at least 6 months, at least 8 months, at least 10 months, at least 12 months or more.

### Preferential Modulation of GABA_{A} Receptor Subtypes

As indicated above, methods of the invention may include providing a composition containing a compound of Formula (I) at a therapeutically effective amount to preferentially modulate one or more GABA_{A} receptor subtypes over other GABA_{A} receptor subtypes. GABA_{A} receptors are ligand-gated ion channels that selectively allow Cl− ions to pass through the plasma membrane upon binding of GABA. GABA_{A} receptors are expressed in neurons throughout the central nervous system (CNS), Leydig cells, placenta, immune cells, liver, bone growth plates, and other endocrine tissues.

Structurally, GABA_{A} receptors are pentamers that include five polypeptide subunits. The polypeptide subunits are encoded by 19 genes that are grouped as follows based on sequence similarity: α(1-6), β(1-3), γ(1-3), δ, ε, θ, π, and ρ(1-3). Most subtypes are heteropentamers that include two copies of one type of α subunit, two copies of one type of β subunit, and one copy of one type of γ, δ, ε, θ, or π subunit; other subtypes are homopentamers or heteropentamers of ρ subunits. Known subtypes of GABA_{A} receptors include α1β1γ2, α1β2γ2, α1β3γ2, α2β1γ2, α2β2γ2, α2β3γ2, α3β1γ2, α3β2γ2, α3β3γ2, α4β1γ2, α4β36, α4β3γ2, α5β1γ2, α5β2γ2, α5β3γ2, α6β1γ2, α6β2γ2, and α6β3γ2. GABA_{A} receptor subtypes vary among tissue types and anatomical regions of the CNS, and subtypes may be associated with specific functions. In addition, GABA_{A} receptor subtypes may vary between normal and malignant cells of the same tissue type.

The active site of a GABA_{A} receptor is the binding site for GABA and for drugs such as muscimol, gaboxadol, and bicuculline. GABA_{A} receptors also have several allosteric binding sites that are the targets of other drugs, including benzodiazepines, nonbenzodiazepines, neuroactive steroids, barbiturates, ethanol, inhaled anaesthetics, and picrotoxin. Thus, the activity of GABA_{A} receptors is controlled by binding of molecules to both the active and allosteric binding sites. The structure, function, and regulation of GABA_{A} receptors are known in the art and described in, for example, Sigel E., and Steinmann, M. E., Structure, Function, and Modulation of GABA_{A} Receptors, J. Biol. Chem. 287:48 pp. 40224-402311 (2012), doi: 10.1074/jbc.R112.386664, the contents of which are incorporated herein by reference.

The compositions of the invention may preferentially potentiate the activity of one or more GABA_{A} receptor subtypes, such as those described above, relative to other GABA_{A} receptor subtypes. In certain embodiments, the compositions preferentially potentiate the activity of α4β3δ receptors compared to α1β2γ2 receptors. For example, as shown herein, isomerically pure compositions of CV-10155 preferentially modulate GABA_{A} receptors of the α4β3δ subtype compared to receptors of the α1β2γ2 subtype

The compositions of the invention may potentiate one or more GABA_{A} receptor subtypes by any mechanism. For example, and without limitation, the isomerically pure form a compound may potentiate a GABA_{A} receptor by allosteric modulation, activation, or inhibition. The allosteric modulation may be positive or negative.

The preferential activity of a composition on one or more GABA_{A} receptor subtypes as compared to other GABA_{A} receptor subtypes may be measured by any suitable means. Activity may be measure using in vitro assays or in vivo assays. For example and without limitation, methods of measuring the effect of modulators on GABA_{A} receptor activity include anticonvulsant assays, binding assays, fluorescence membrane potential assays, immune response assays, intracranial self-stimulation assays patch clamps assays, proliferation assays receptor occupancy assays seizure induction assays, e.g., using pentylenetetrazol (PTZ) or maximal electroshock (IVIES), and survival assays. Such assays are known in the art and described in, for example, International Publication No. WO 2016/061527; Ghisdal P., et al., Determining the relative efficacy of positive allosteric modulators of the GABA_{A} receptor: design of a screening approach, J Biomol Screen. 2014 March; 19(3):462-7. doi: 10.1177/1087057113501555, Epub 2013 Aug. 29; Tian J., et al., Clinically applicable GABA receptor positive allosteric modulators promote β-cell replication, Sci Rep. 2017 Mar. 23; 7(1):374. doi: 10.1038/s41598-017-00515-y; and Tian J., et al., A Clinically Applicable Positive Allosteric Modulator of GABA Receptors Promotes Human β-Cell Replication and Survival as well as GABA's Ability to Inhibit Inflammatory T Cells, J Diabetes Res. 2019 Feb. 26; 2019:5783545, doi: 10.1155/2019/5783545, the contents of each of which are incorporated herein by reference.

The preferential activity of a composition on one or more GABA_{A} receptor subtypes as compared to other GABA_{A} receptor subtypes may be expressed by any suitable means. For example and without limitation, the preferential activity may be indicated by a comparison of EC50 values or binding affinity values.

In certain embodiments, compositions of the invention have an EC50 for α4β3δ GABA_{A} receptors that is lower than the EC50 for α1β2γ2 GABA_{A} receptors. The EC50 for α4β3δ GABA_{A} receptors may be lower than the EC50 for α1β2γ2 GABA_{A} receptors by about 2-fold, about 3-fold, about 4-fold, about 5-fold, about 6-fold, about 7-fold, about 8-fold, about 10-fold, about 20-fold, about 50-fold, about 100-fold, about 200-fold, about 500-fold, or about 1000-fold.

In certain embodiments, compositions of the invention have an EC50 for α4β3δ GABA_{A} receptors that is less than about 50%, less than about 40%, less than about 30%, less than about 25%, less than about 20%, less than about 15%, less than about 10%, less than about 5%, less than about 4%, less than about 3%, less than about 2%, less than about 1%, less than about 0.5%, less than about 0.2%, or less than about 0.1% of the EC50 for α1β2γ2 GABA_{A} receptors.

In certain embodiments, compositions of the invention have a binding affinity (which may be expressed, e.g., as a dissociation constant KD) for α4β3δ GABA_{A} receptors that is lower than the binding affinity for α1β2γ2 GABA_{A} receptors. The binding affinity for α4β3δ GABA_{A} receptors may be lower than the binding affinity for α1β2γ2 GABA_{A} receptors by about 2-fold, about 3-fold, about 4-fold, about 5-fold, about 6-fold, about 7-fold, about 8-fold, about 10-fold, about 20-fold, about 50-fold, about 100-fold, about 200-fold, about 500-fold, or about 1000-fold.

In certain embodiments, compositions of the invention have an binding affinity for α4β3δ GABA_{A} receptors that is less than about 50%, less than about 40%, less than about 30%, less than about 25%, less than about 20%, less than about 15%, less than about 10%, less than about 5%, less than about 4%, less than about 3%, less than about 2%, less than about 1%, less than about 0.5%, less than about 0.2%, or less than about 0.1% of the binding affinity for α1β2γ2 GABA_{A} receptors.

In certain embodiments, compositions of the invention have an EC50 for α4β3δ GABA_{A} receptors that is below a defined value. For example and without limitation, the composition may have an EC50 for α4β3δ GABA_{A} receptors that is less than about 1 μM, less than about 500 nM, less than about 400 nM, less than about 300 nM, less than about 200 nM, less than about 100 nM, less than about 50 nM, less than about 25 nM, less than about 10 nM, less than about 5 nM, less than about 2.5 nM, less than about 1 nM, less than about 0.5 nM, less than about 0.25 nM, or less than about 0.1 nM.

In certain embodiments, compositions of the invention have a binding affinity for α4β3δ GABA_{A} receptors below a defined value. For example and without limitation, the composition may have an binding affinity for α4β3δ GABA_{A} receptors that is less than about 1 less than about 500 nM, less than about 400 nM, less than about 300 nM, less than about 200 nM, less than about 100 nM, less than about 50 nM, less than about 25 nM, less than about 10 nM, less than about 5 nM, less than about 2.5 nM, less than about 1 nM, less than about 0.5 nM, less than about 0.25 nM, or less than about 0.1 nM.

### EXAMPLES

### Example 1

The ability of the compound of Formula (I), referred to as CV-10155, and a stereoisomer called SPNC-019 to modulate the activity of GABA_{A} receptors of different subtypes was analyzed. CV-10155 and SPNC-019 have the following structures:

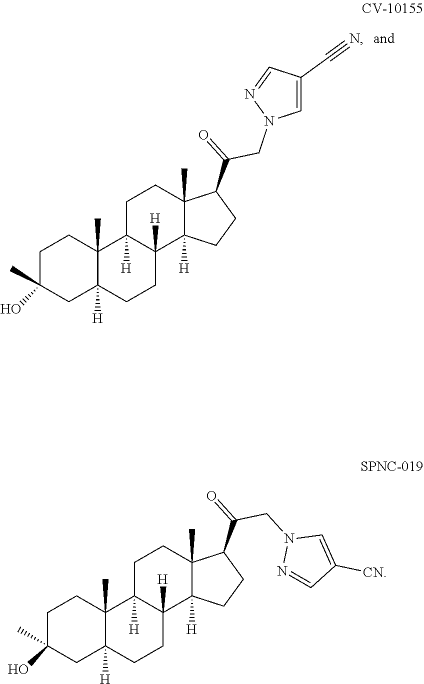

The only structural difference between CV-10155 and SPNC-019 is the stereochemical configuration of the hydroxyl and methyl groups attached to the carbon atom at position 3 of the steroid core.

Cells expressing the indicated GABA_{A} receptor subtype were exposed to gamma-aminobutyric acid in the presence of varying concentrations of either CV-10155 or SPNC-019, calcium flux was measured using a fluorometric imaging plate reader (FLIPR), and EC50 values for compounds were determined. Results are provided in Table 1.

TABLE 1

EC₅₀ (M)

| | | | |
|---|---|---|---|
| GABA_{A} | Compound | CV-10155 | SPNC-019 |

| | | |
|---|---|---|
| α1β1γ2 | 5.21E−07 | — |
| α1β2γ2 | 8.39E−07 | — |
| α1β3γ2 | 5.20E−07 | — |
| α2β1γ2 | 2.53E−07 | — |
| α2β2γ2 | 2.13E−07 | — |
| α2β3γ2 | 2.96E−07 | — |
| α3β1γ2 | 9.46E−07 | — |
| α3β2γ2 | 1.82E−06 | — |
| α3β3γ2 | 2.73E−07 | — |
| α4β1γ2 | 2.04E−07 | — |
| α4β3δ | 1.06E−07 | — |
| α4β3γ2 | 1.33E−06 | — |
| α5β1γ2 | 7.62E−07 | — |
| α5β2γ2 | 3.59E−07 | 1.198e−006 |
| α5β3γ2 | 1.30E−06 | 1.805e−006 |
| α6β1γ2 | 3.59E−07 | — |
| α6β2γ2 | 1.10E−06 | — |
| α6β3γ2 | 2.45E−07 | 1.766e−006 |

—value not measurable

CV-10155 showed some level of positive allosteric modulating activity in all of the GABA_{A} receptor subtypes tested. In contrast, SPNC-019 had no modulating activity in 15 of the 18 GABA_{A} receptor subtypes tested. Thus, the results show that a change in the stereochemistry of a single chiral center of a steroid-based compound dramatically alters ability of the molecule to modulate GABA_{A} receptor activity. The results further indicate that the isomeric purity of compositions containing compounds of Formula (I) greatly impacts the utility of such compositions as therapeutic agents.

### Example 2

The ability of various steroids to compete with t-butylbicyclophosphorothionate (TBPS), a ligand for the picrotoxin binding site of GABA_{A} receptors, was analyzed in International Publication No. WO 2016/061527. WO 2016/061527, pages 215-227. Compounds were assayed for binding to GABA receptors in membranes isolated from the cortices of rat brains. WO 2016/061527, page 216.

Among the steroids analyzed was Compound 10, which has the following structure:

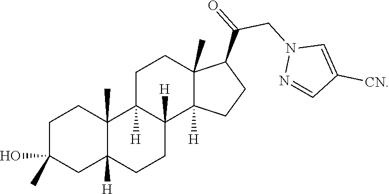

WO 2016/061527, page 106. Compound 10 is identical to the structure of Formula (II) and is a stereoisomer of the structure of Formula (I). Compound 10/Formula (II) and Formula (I) are stereoisomers that differ only in the configuration of the hydrogen atom bonded to the carbon atom at position 5: Compound 10/Formula (II) has a 5β configuration, whereas Formula (I) has a 5α configuration.

Another steroid analyzed in WO 2016/061527 was Compound 121, which has the following structure:

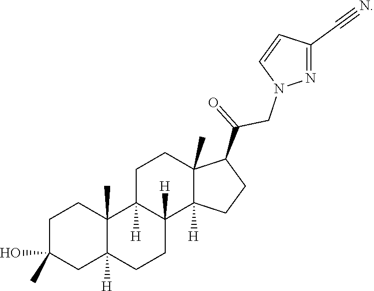

WO 2016/061527, page 150. Compound 121 is a regioisomer of the structure of Formula (I). Compound 121 and Formula (I) differ only in the positioning of the cyano substituent on the pyrazole ring: Compound 121 is substituted at the 3 position of the pyrazole ring, whereas Formula (I) is substituted at the 4 position of the pyrazole ring.

Compound 10 and Compound 121 are isomers that have two structural differences: the stereochemical configuration at carbon 5, and the position of the cyano substituent on the pyrazole ring.

Results of the analysis are provided in Table 1 of WO 2016/061527. WO 2016/061527, pages 217-227. Compound 10 has an IC₅₀ of <10 nM in the TBPS displacement assay, whereas Compound 121 has an IC₅₀ of 10-50 nM. WO 2016/061527, pages 217 and 221.

These results show that subtle structural differences in a steroid can drastically affect binding of the molecule to GABA_{A} receptors.

### Example 3

The pharmacological efficacy of various steroids for α1β2γ2 GABA_{A} receptors and α4β3δ GABA_{A} receptors and was analyzed in International Publication No. WO 2016/061527. WO 2016/061527, pages 227-231. Compounds were tested for the ability to modulate GABA-mediated currents at a submaximal dose of agonist in LTK cells stably transfected with α1β2γ2 subunits and in CHO cells transiently transfected with α4β3δ subunits WO 2016/061527, pages 227-228. Cells were incubated with GABA at 2 μM, which is the EC₂₀ for GABA, and 0.01 μM, 0.1 μM, 1 μM, or 10 μM steroid. WO 2016/061527, pages 227-228.

Results of the analysis are provided in Table 2 of WO 2016/061527. WO 2016/061527, pages 229-231. Results are presented as the relative potentiation of GABA-mediated conductance in the presence of 10 μM steroid compared to GABA-mediated conductance in the absence of steroid. WO 2016/061527, page 228. Compound 121 at 10 μM displayed an efficacy of >500% for both α1β2γ2 GABA_{A} receptors and α4β3δ GABA_{A} receptors. WO 2016/061527, page 229.

The results show that a regioisomer of Formula (I) displays no preferential modulation of α4β3δ GABA_{A} receptors over α1β2γ2 GABA_{A} receptors. In particular, a compound that differs from Formula (I) only by the positioning of the cyano substituent on the pyrazole ring has comparable efficacy on the two GABA_{A} receptor subtypes. Thus, the data give no indication that compositions containing a compound of Formula (I) can preferentially modulate α4β3δ GABA_{A} receptors over α1β2γ2 GABA_{A} receptors or that such compositions can be administered at concentrations that modulate α4β3δ GABA_{A} receptors but not α1β2γ2 GABA_{A} receptors. Consequently, nothing from the results suggests that compositions containing the compound of Formula (I) would be useful for treatment of conditions in which potentiation of α4β3δ GABA_{A} receptors but not α1β2γ2 GABA_{A} receptors is beneficial.

In contrast, the data provided in Example 1 show that the compound of Formula (I) is substantially more active on α4β3δ GABA_{A} receptors than on α1β2γ2 GABA_{A} receptors. Taken together, the results in the Examples demonstrate that subtle structural differences in a steroid affect the ability of the molecule to potentiate specific subtypes of GABA_{A} receptors. Therefore, it follows from the results that the isomeric purity of steroid compositions can influence receptor subtype specificity and thus the utility of such compositions as therapeutic agents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof

What is claimed is:

1. A method of treating fibromyalgia in a human subject, the method comprising providing to a human subject having a fibromyalgia a compound of Formula (I):

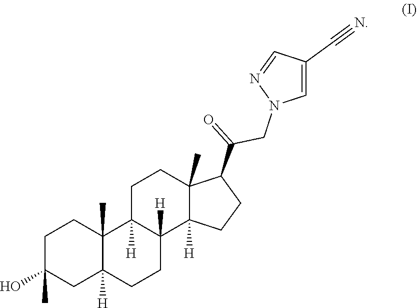

2. The method of claim 1, wherein the compound of Formula (I) is provided in a composition comprising the compound of Formula (I).

3. The method of claim 2, wherein the fibromyalgia is associated with a symptom selected from the group consisting of chronic pain, allodynia, hyperalgesia, and fatigue.

4. The method of claim 3, wherein treating fibromyalgia comprises treating at least one of chronic pain, allodynia, hyperalgesia, and fatigue.

5. The method of claim 4, wherein treating fibromyalgia comprises reducing fibromyalgia-associated pain by at least 50%.

6. The method of claim 4, wherein treating fibromyalgia comprises reducing fibromyalgia-associated pain by at least 60%.

7. The method of claim 4, wherein the fibromyalgia is associated with a symptom selected from the group consisting of depression, anxiety, stiffness, sleep disorders, difficulty concentrating, memory loss, headaches, vision problems numbness, temporomandibular joint syndrome, ringing in the ears, digestive problems, including irritable bowel and bladder disorders, restless leg syndrome, skin sensitives and rashes, and dry eyes and mouth.

8. The method of claim 7, wherein treating fibromyalgia comprises treating one or more symptom selected from the group consisting of depression, anxiety, stiffness, sleep disorders, difficulty concentrating, memory loss, headaches, vision problems numbness, temporomandibular joint syndrome, ringing in the ears, digestive problems, including irritable bowel and bladder disorders, restless leg syndrome, skin sensitives and rashes, and dry eyes and mouth.

9. The method of claim 2, wherein the composition is provided orally.

11. The method of claim 2, wherein the composition is provided in a single dose per day.

12. The method of claim 2, wherein the composition is provided in multiple doses per day.

13. The method of claim 2, further comprising administering one or more additional active agents selected from the group consisting of antidepressants, anti-inflammatories, muscle relaxants, antibiotics, mood stabilizers, antipsychotics, serotonin receptor antagonists, serotonin receptor agonists, pain relievers, stimulants, NMDA receptor ligands, serotonin reuptake inhibitor antidepressants, serotonin-norepinephrine reuptake inhibitors, and anticonvulsants.

14. The method of claim 2, wherein the subject is a human female subject.
